(19) United States
(12) Patent Application Publication
(10) Pub. No.: US 2026/0058017 A1
(43) Pub. Date: Feb. 26, 2026

(54) SMART DENTAL FLOSSER

(71) Applicant: University of Southern California, Los Angeles, CA (US)

(72) Inventors: Maral Mousavi, Los Angeles, CA (US); Sina Khazaee Nejad Gharahtekan, Los Angeles, CA (US); Meisam Razaviyayn, Los Angeles, CA (US)

(21) Appl. No.: 19/304,129

(22) Filed: Aug. 19, 2025

Related U.S. Application Data
(60) Provisional application No. 63/686,303, filed on Aug. 23, 2024.

(51) Int. Cl. G16H 50/20; A61B 5/00; A61B 10/00; A61C 15/04

(52) U.S. Cl. CPC G16H 50/20; A61B 5/7267; A61B 10/0051; A61C 15/046

(57) ABSTRACT

Disclosed herein are devices comprising a) a holder comprising one or more yarns comprising one or more filaments and a receptacle; wherein the one or more yarns is positioned in a holder such that a portion of the one or more yarns is exposed and is configured to be placed in an oral cavity between two or more teeth; wherein the one or more yarns and the receptacle are in fluid communication, wherein the one or more yarns is configured to collect, store and/or transfer one or more fluids from and/or to the oral cavity; and wherein the device is configured to analyze the health of the oral cavity and/or deliver a treatment to the oral cavity. Also disclosed are kits comprising the same and methods of making and using such devices.

### CROSS-REFERENCE TO RELATED APPLICATIONS

This application claims the benefit of U.S. Provisional Application No. 63/686,303, filed Aug. 23, 2024, the content of which is incorporated herein by reference in its entirety.

### TECHNICAL FIELD

The present invention generally relates to devices configured to evaluate an oral cavity, diagnose and monitor potential disorders, and treat such if needed. Also disclosed are kits comprising such devices and methods of making the same.

### BACKGROUND

Gingivitis and periodontitis are widespread public health concerns. They are chronic inflammatory diseases that are characterized by the destruction of dentition-supporting tissues, progressive alveolar bone resorption, and eventual tooth loss. The disease progresses in stages, while in early stages (gingivitis), it can be reversed. Periodontitis is one of the most prevalent chronic inflammatory diseases, affecting 30-35% of the global population, 47.2% of adults aged 30 years and older in the US, and 70% of adults 65 years and older. Its prevalence has caused the Centers for Disease Control and Prevention (CDC) to label it a strategic focus for nearly two decades. A study by the British Dental Association showed that 90% of the global population will experience some degree of gum recession and loss of connective tissue in their life. Apart from causing gum inflammation, bleeding, tissue and bone recession, and eventual tooth loss, periodontitis has also been associated with several systemic diseases, such as atherosclerosis, diabetes, cancers, and Alzheimer's disease. Periodontal inflammation can trigger the pathogenesis of systematic chronic illnesses. The direct costs (e.g., treatment costs) and dental health expenditures associated with periodontal disease were estimated to be $3.5 billion annually in the US. The actual burden associated with this disease is much higher ($150 billion annually), as there are indirect costs from the consequences of periodontitis, such as absenteeism from work and productivity losses.

Unfortunately, there are pervasive disparities in the prevalence of periodontal disease among US adults of different ethnicities, education levels, and incomes. These differences are associated with disparities in access to regular preventive dental care, and socioeconomic factors.

A healthy gum is attached to the teeth, is pink colored, and is supported by dense bone structure underneath. The start of gingivitis and periodontitis is linked to poor oral hygiene, and plaque and bacteria build up close to the gingival sulcus. This chronic multifactorial disease then progresses in a positive destructive loop between pathogenic organisms and the body's immune response and inflammation, leading to tissue and bone damage.

Currently, periodontitis is screened for during regular dental checkups with visual verification (inflammation and swelling in the gum), measuring the depth of the periodontal pocket using dental probes, and, in some cases, with X-ray imaging. There are three limitations to these state-of-the-art detection methods: (i) they are unable to identify the disease early before visible inflammation and gum recession occurs (when treatment is most effective), (ii) they cannot be performed frequently, and can only be administered when patients come in for a check-up (semi-annual if patients comply), and (iii) they present an economic barrier for low-income patients who have limited or no dental insurance (ironically the population disproportionately affected by periodontitis). The most common method of screening is using a dental probe to measure the depth of the periodontal pocket; this method is prone to discrepancies related to pressure applied by the nurse or dentist during measurements.

Some experimental early screening commercial products have recently been commercialized for chairside testing (the Omnigene Diagnostics Test, the Evalusite kit, and the PerioSafe kit). These kits' function is based on lateral flow assays, sandwich immunoassays, and nucleic acid hybridization. These tests were used in clinical settings but are not suitable for rapid diagnostics. For example, the Omnigene Diagnostics assay is too slow for chairside testing (e.g., a few hours to days); the sensitivity of the Evalusite kits is too low to satisfy clinical needs; and the lateral flow PerioScan kit can only qualitatively detect the disease severity. Moreover, most of these tests require reagent addition, mixing, and washing steps, which are time-consuming and require some skill for operation. In addition, these kits are expensive and often require an additional expense of a doctor or hygienist visit.

Accordingly, a need exists for affordable devices capable of evaluating the health of the oral cavity, as well as diagnosing and possibly treating various oral cavity ailments. These needs and other needs are at least partially satisfied by the present disclosure.

### SUMMARY

The present disclosure is directed to a device comprising a) a holder comprising one or more yarns comprising one or more filaments and a receptacle; wherein the one or more yarns is positioned in a holder such that a portion of the one or more yarns is exposed and is configured to be placed in an oral cavity between two or more teeth; wherein the one or more yarns and the receptacle are in fluid communication, wherein the one or more yarns is configured to collect, store and/or transfer one or more fluids from and/or to the oral cavity; and wherein the device is configured to analyze the health of the oral cavity and/or deliver a treatment to the oral cavity.

In certain aspects, the one or more filaments of the present disclosure can be hydrophilic. In certain aspects, the one or more filaments of the present disclosure can be conductive.

In certain aspects of the instant disclosure, the holder can further comprise one or more hydrophobic filaments positioned in the holder such that a portion of the one or more hydrophobic filaments is exposed and is configured to be placed in an oral cavity between two or more teeth together with the one or more yarns, wherein the one or more filaments are not in fluid communication with the receptacle, and wherein the one or more hydrophobic filaments provide mechanical reinforcement to the one or more yarns.

In certain aspects of the instant disclosure, the device can comprise at least one microfluidic channel that is in fluid communication with the one or more yarns. In certain aspects of the instant disclosure, the device can further comprise at least one wicking element, wherein the at least one wicking element is in fluid communication with the one or more yarns and/or with the at least one microfluidic channel if present.

In still certain aspects, the device disclosed herein can be electronically connected to a processing unit configured to evaluate and/or indicate a characteristic of at least one property of a biofluid present in the oral cavity. In certain aspects, the processing unit can comprise a detector.

The present invention is also directed to a kit comprising any of the devices disclosed above.

In still further aspects, the disclosure is directed to a method of making of any of the disclosed herein devices.

Yet still in further aspects, the disclosure is directed to a method comprising positioning any of the disclosed herein devices within an oral cavity such that the exposed portion of the one or more yarns is placed between two or more teeth; collecting a biofluid from the oral cavity to determine the health of oral cavity and/or delivering a treatment fluid to the oral cavity.

Also disclosed herein is a method comprising receiving a plurality of biomarker measurements, wherein each of the plurality of biomarker measurements correspond to a patient of a plurality of patients; receiving a plurality of known disease diagnoses for each of the plurality of patients; creating a training set comprising the plurality of biomarker measurements and the plurality of known disease diagnoses; and training a machine learning model using the training set, wherein the trained machine learning model is configured to predict a disease diagnosis for a patient based on a biomarker measured from the patient.

Also disclosed is a method of predicting a disease state of a patient comprising: receiving a trained machine learning model configured to predict a disease diagnosis for a patient based on a biomarker measured from the patient; measuring the biomarker of the patient using an electrochemical impedance spectroscopy device; inputting the biomarker of the patient into the trained machine learning model; and receiving, from the trained machine learning model, a predicted disease diagnosis.

Also disclosed is a system comprising any of the devices disclosed herein and at least one processing unit.

Also disclosed is a system for predicting a disease state of a patient, the system comprising: a computing device operably coupled to the device of any one of the examples herein, wherein the computing device comprises at least one processor and memory, the memory having computer-executable instructions stored thereon that, when executed by the at least one processor, cause the at least one processor to: perform the method of any one of the examples herein.

Additional aspects of the disclosure will be set forth, in part, in the detailed description, figures, and claims that follow, and in part, will be derived from the detailed description or can be learned by practice of the invention. It is understood that both the foregoing general description and the following detailed description are exemplary and explanatory only and are not restrictive of the invention as disclosed.

### BRIEF DESCRIPTION OF DRAWINGS

FIG. 1 depicts a schematic of an exemplary device according to some aspects of the disclosure.

FIG. 2 depicts a schematic of an exemplary device according to some aspects of the disclosure.

FIG. 3 depicts a schematic of an exemplary device according to some aspects of the disclosure.

FIGS. 4A-4C depict schematics of an exemplary device according to some aspects of the disclosure.

FIGS. 5A-5C depict schematics of an exemplary device according to some aspects of the disclosure.

FIG. 6 depicts a photograph of an exemplary method of using an exemplary device according to some aspects of the disclosure.

FIG. 7 depicts an exemplary handheld reading device and an exemplary response.

FIGS. 8A-8C show a schematic of an exemplary device according to some aspects of the disclosure (FIG. 8A) and exemplary modification of the one or more yarns in the exemplary device (FIGS. 8B-8C).

FIG. 9 depicts a diagram of a portable multichannel analyzer according to some aspects of the disclosure.

FIGS. 10A-10B depict a method of sensor fabrication using laser engraving on a polymer film (A) and a scanning electron microscope image of the sensor surface that is modified with nanoparticles (B).

FIGS. 11A-11B depict the electrochemical detection of biomarkers with the sensors, where changes in electrical current are proportional to biomarker concentration.

FIG. 12 depicts an exemplary Machine learning model. Patients will be categorized into healthy gingivitis, generalized early, and moderate/severe periodontitis. Traditional diagnoses for training data are performed according to state-of-the-art protocols.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the singular forms “a,” “an,” and “the” include plural referents unless the context clearly dictates otherwise. Thus, for example, reference to a “device” or an “electrode” includes aspects having two or more such devices or electrodes unless the context clearly indicates otherwise.

In still further aspects and as used herein, the terms “wicking element” and “microfluidic channel” can be used interchangeably. The wicking element is defined by its ability to pull fluid without the need for a pump. It is understood that the wicking element can be described as a microfluid channel. However, it is also understood that not all microfluid channels can have a wicking property (pulling fluid without needing a pumping element or any other external force).

It is appreciated that certain features of the disclosure, which are, for clarity, described in the context of separate aspects, can also be provided in combination with a single aspect. Conversely, various features of the disclosure, which are, for brevity, described in the context of a single aspect, can also be provided separately or in any suitable subcombination.

As used herein, the terms “optional” or “optionally” mean that the subsequently described event or circumstance may or may not occur and that the description includes instances where said event or circumstance occurs and instances where it does not.

Throughout the description and claims of this specification, the word “comprise” and other forms of the word, such as “comprising” and “comprises,” means including but not limited to, and are not intended to exclude, for example, other additives, segments, integers, or steps. Furthermore, it is to be understood that the terms comprise, comprising, and comprises as they relate to various aspects, elements, and features of the disclosed invention also include the more limited aspects of “consisting essentially of” and “consisting of.”

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The term “or” means “and/or.” Recitation of ranges of values is merely intended to serve as a shorthand method of referring individually to each separate value falling within the range unless otherwise indicated herein, and each separate value is incorporated into the specification as if it were individually recited herein. The endpoints of all ranges are included within the range and are independently combinable. All methods described herein can be performed in a suitable order unless otherwise indicated herein or otherwise clearly contradicted by context.

The expressions “ambient temperature” and “room temperature” as used herein are understood in the art and refer generally to a temperature from about 20° C. to about 35° C.

Notwithstanding that the numerical ranges and parameters setting forth the broad scope of the disclosure are approximations, the numerical values set forth in the specific examples are reported as precisely as possible. Any numerical value, however, inherently contains certain errors that necessarily result from the standard deviation found in their respective testing measurements. Furthermore, when numerical ranges of varying scope are set forth herein, it is contemplated that any combination of these values, inclusive of the recited values, may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

It is understood that the term “between,” when used in the context of ranges, includes the bordering values of the range. For example, a range described as being between 10 and 15 includes both 10 and 15 unless described otherwise.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase ‘x, y, z, or greater’ should be interpreted to include the specific ranges of ‘x,’ ‘y,’ ‘z,’ ‘about x,’ ‘about y,’ and ‘about z’ as well as the ranges of ‘greater than x,’ greater than y,′ ‘greater than z,’ or ‘greater than about x,’ greater than about y,′ ‘greater than about z.’ In addition, the phrase “‘x’ to ‘y’,” where ‘x’ and ‘y’ are numerical values, also includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Recitation of ranges of values herein is merely intended to serve as a shorthand method of referring individually to each separate value recited or falling within the range unless otherwise indicated herein, and each separate value is incorporated into the specification as if it were individually recited. Ranges provided herein are understood to be shorthand for all of the values within the range. For example, a range of 1 to 50 is understood to include any number, or combination of numbers, from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or sub-ranges from the group consisting of 10-40, 20-50, 5-35, etc. Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

In still further aspects, when the specific values are disclosed between two end values, it is understood that these end values can also be included.

In still further aspects, when the range is given, and exemplary values are provided, it is understood that any ranges can be formed between any exemplary values within the broadest range.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

Spatially relative terms, such as “beneath,” “below,” “lower,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one embodiment to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the term “substantially” means that the subsequently described event or circumstance completely occurs or that the subsequently described event or circumstance generally, typically, or approximately occurs.

Still further, the term “substantially” can, in some aspects, refer to at least about 80%, at least about 85%, at least about 90%, at least about 91%, at least about 92%, at least about 93%, at least about 94%, at least about 95%, at least about 96%, at least about 97%, at least about 98%, at least about 99%, or about 100% of the stated property, characteristic, component, composition, or other condition for which substantially is used to characterize or otherwise quantify an amount.

As used herein, the term “substantially,” in, for example, the context “substantially identical” or “substantially similar” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

As used herein, the term “measured” can refer in some aspects an exact or precise quantitative measurement, while in other aspects, it can also refer to measuring relative amounts, rates of change, or qualitative data. It is understood that any value that is measured can be presented in any form. In certain aspects, the data can be presented as a final concentration, as a range, as a qualitative response of “yes” or “no,” or any other form that conveys any sought information.

Similarly, when the disclosure refers to a device configured to indicate the presence of the at least one characteristic of the fluid, it is understood that the term “presence” can include numerical values, visual representation, qualitative response, etc. It is further understood that this term, as used in the specified context, can also include a simple presence of the indicated property, relative amounts of the indicated property, ranges of various amounts, calibrated amounts against different reagents or components, or even precise amounts when possible. In yet further aspects, the term “presence” can indicate ranges such as, for example, “low,” “medium,” and/or “high.” It can also indicate any ranges in between. This indication can be done visually by the color scale, or it can also show specific wording or numbers as desired. It is further understood that any representation that helps the device's wearer estimate the amount of the indicated property is included in this disclosure.

Numerous other general-purpose or special-purpose computing device environments or configurations can be used. Examples of well-known computing devices, environments, and/or configurations that can be suitable for use include, but are not limited to, personal computers, server computers, handheld or laptop devices, smartphones, multiprocessor systems, microprocessor-based systems, network personal computers (PCs), minicomputers, mainframe computers, embedded systems, distributed computing environments that include any of the above systems or devices, and the like.

Computing devices, as disclosed herein, can contain communication connection(s) that allow the device to communicate with other devices if desired. Computing devices can also have input device(s) such as a keyboard, mouse, pen, voice input device, touch input device, etc. Output device(s) such as a display, speakers, printer, etc., can also be included. All these devices are well-known in the art and need not be discussed at length here.

Computer-executable instructions, such as program modules being executed by a computer, can be used. Generally, program modules include routines, programs, objects, components, data structures, etc., that perform particular tasks or implement particular abstract data types. Distributed computing environments can be used where tasks are performed by remote processing devices that are linked through a communications network or other data transmission medium. In a distributed computing environment, program modules and other data can be located in both local and remote computer storage media, including memory storage devices.

In its most basic configuration, a computing device typically includes at least one processing unit and memory. Depending on the exact configuration and type of computing device, memory can be volatile (such as random-access memory (RAM)), non-volatile (such as read-only memory (ROM), flash memory, etc.), or a combination of both.

Computing devices can have additional features/functionality. For example, a computing device can include additional storage (removable and/or non-removable), including, but not limited to, magnetic or optical disks or tape.

Computing device typically includes a variety of computer-readable media. Computer-readable media can be any available media that the device can access, including both volatile and non-volatile media, as well as removable and non-removable media.

Computer storage media include volatile and non-volatile, and removable and non-removable media implemented in any method or technology for the storage of information, such as computer-readable instructions, data structures, program modules, or other data. Memory, removable storage, and non-removable storage are all examples of computer storage media. Computer storage media include but are not limited to, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device. Any such computer storage media can be part of a computing device.

Computing devices, as disclosed herein, can contain communication connections(s) that allow the device to communicate with other devices. The connection can be wireless or wired. Computing devices can also have input device(s) such as a keyboard, mouse, pen, voice input device, touch input device, etc. Output device(s) such as a display, speakers, printer, etc., can also be included. All these devices are well-known in the art and need not be discussed at length here.

It should be understood that the various techniques described herein can be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, can take the form of program code (i.e., instructions) embodied in tangible media, such as CD-ROMs, hard drives, or any other machine-readable storage medium where, when the program code is loaded into and executed by a machine, such as a computer, the machine becomes an apparatus for practicing the presently disclosed subject matter.

Also, it is further understood that the devices disclosed herein can be in communication with other computerized devices by various means. In certain aspects, the sensors can transfer the information to the computerized devices with cameras or any other devices configured to capture a visual response of the measuring device. In yet other aspects, where the measuring device response is a change of color, it is understood that the change of color can occur in the visual spectra of the light.

It is further understood, however, that the measuring response device can also occur in UV or IR spectra. In such aspects, the response can be further evaluated by additional means, and the final result can be presented to the device wearer. It is also understood that the response of the devices disclosed herein can include photo fluorescence, fluorescence, and/or luminescent responses.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

Moreover, for the sake of simplicity, the attached figures cannot show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

The present invention may be understood more readily by reference to the following detailed description of various aspects of the invention and the examples included therein, and to the Figures and their previous and following description.

### Device

In some aspects, disclosed herein is a device comprising: a holder comprising one or more yarns comprising one or more filaments and a receptacle. In exemplary and unlimiting aspects, the one or more yarns are positioned in the holder such that a portion of the one or more yarns is exposed and is configured to be placed in an oral cavity between two or more teeth. In such exemplary aspects, the one or more yarns and the receptacle are in a fluid communication, wherein the one or more yarns are configured to collect, store, and/or transfer one or more fluids from and/or to the oral cavity; wherein the device is configured to analyze the health of the oral cavity and/or deliver a treatment to the oral cavity.

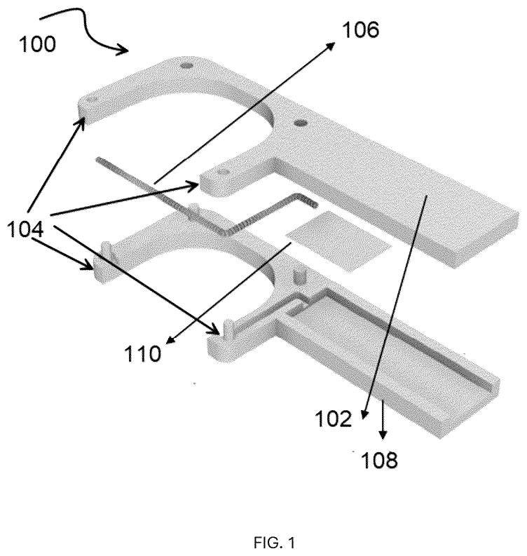

The exemplary device 100 is shown in FIG. 1. The holder 102 can comprise one or more yarns 106 and at least one receptacle 108. In certain aspects, the holder 102 can comprise a portion 104 configured to hold the exposed portion of the one or more yarns 106. This portion 104 is configured to be conveniently inserted into the oral cavity such that the exposed portion of one or more yarns is positioned between two or more teeth. In this exemplary and unlimiting aspect, the exposed portion of one or more yarns can be positioned within the holder, for example, under a predetermined tension to allow convenient insertion into the oral cavity.

In certain aspects, the holder 102 can have any shape that would allow convenience for the user. The holders shown in FIGS. 1-6 and 8A are only exemplary. It is understood that the holder itself can be made by any method known in the art. For example, it can be formed from a mold, or it can be 3-D printed, or it can be a combination of modeling and 3D printing. In the figures shown here, for example, the holder can be formed by 3D printing of two or more parts that are then combined with other components of the device to form the final holder and the device. In still further aspects, the device can have a shape and size convenient for a user.

In still further aspects, the receptacle can have any shape and desired volume. It is understood that the volume of the receptacle should be effective to collect and/or hold enough fluid for the analysis or treatment if needed. In certain aspects, the receptacle can have a volume of 0.1 μL to 15 μL, including exemplary values of 0.5 μL, 1 μL, 2 μL, 3 μL, 4 μL, 5 μL, 6 μL, 7 μL, 8 μL, 9 μL, 10 μL, 11 μL, 12 μL, 13 μL, and 14 μL. It is understood that the volume of the receptacle can have any value that falls between any two foregoing values or within a range formed by any two foregoing values. For example, the volume of the receptacle can be 0.1 μL to 10 μL, 0.1 μL to 8 μL, 0.1 μL to 6 μL, 0.1 μL to 4 μL, 0.5 μL to 15 μL, 1 μL to 15 μL, 2 μL to 15 μL, 4 μL to 15 μL, 6 μL to 15 μL, 8 μL to 15 μL, 10 μL to 15 μL, and so on.

In still further aspects, one or more fluids can comprise any fluid of interest. For example, in one aspect, a first fluid of the one or more fluids can be a biofluid transferred from the oral cavity to the receptacle. In other words, in certain aspects, the first fluid is the fluid that is tested to determine the health of the oral cavity. For example, and without limitations, the biofluid can be a gingival crevicular (GC) fluid, saliva, or a combination thereof. In still further aspects, the biofluid is the GC fluid. In other aspects, the biofluid is saliva. In yet other aspects, the biofluid is a mixture of GC fluid and saliva. In still further aspects, the first fluid can comprise any of the disclosed above biofluids and an additional agent. In such aspects, this additional agent can comprise a pharmacologically active ingredient (a medication, a topical treatment, growth hormones, enzyme inhibitors, and the like), food residues, nutrients, oral cavity debris, and so on. It is understood that the first fluid that is collected in the oral cavity and transferred to the analysis can comprise components that are not biofluid.

In still further aspects, a second fluid of the one or more fluids is a pharmacologically active agent positioned in the receptacle. In such exemplary aspects, this second fluid is delivered from the receptacle to the oral cavity. For example, in certain aspects, the device can be used for collecting and transferring the first fluid to the receptacle for the detection of specific biomarkers and evaluation of whether any ailment exists. Yet, in other exemplary aspects, the device can be used for delivering the second fluid from the receptacle to the oral cavity for a specific treatment. Yet in still further aspects, the device can be used to collect the first fluid for analysis and deliver the second fluid for treatment.

In certain aspects, the device can combine two or more receptacles. Yet in other aspects, the device can have one receptacle that has two or more compartments. In such aspects, the compartments or receptacles can be substantially isolated from each other, or they can be in electric and/or fluidic communication with each other. For example, and without limitations, one receptacle (or a compartment) can comprise sensing elements for detecting the predetermined biomarkers and evaluating the oral cavity health, yet another receptacle (or compartment) can comprise a pharmaceutically active agent or any other active agents that can be used to treat the oral cavity if needed. Yet in still further aspects, at least one of the receptacles can be detachable. Yet, in other aspects, both receptacles can be detachable.

It is understood that the term “pharmaceutically active” ingredients broadly covers any medications, vitamins, hormones, and enzyme inhibitors. In still further aspects, the active ingredients can also comprise minerals, nutrients, debris, and/or plaque-removing reagents, stabilizers, and so on.

In some aspects, and as disclosed above, the one or more yarns comprise one or more filaments. In other words, the yarn can be monofilament or multifilament. If the yarn is multifilament, any number of filaments from 2 to 100, 2-80, 2-50, 2-30, 2-20, 2-10, 2-5, 5-100, 10-100, 20-100, 40-100, 80-100, and so on can be present.

In still further aspects, the one or more filaments in the one or more yarns can have a diameter of 1-10 microns, including 2 microns, 3 microns, 4 microns, 5 microns, 6 microns, 7 microns, 8 microns, and 9 microns. In still further aspects, the diameter of the one or more filaments can have any value that falls between any two foregoing values or within a range formed by any two foregoing values. For example, the diameter can be 1-2 microns, 1-3 microns, 1-5 microns, 1-8 microns, and so on. In yet other aspects, the one or more filaments can have a diameter of less than 5 microns, less than 4 microns, less than 3 microns, or 1-5 microns, 1-4 microns, 1-3 microns, 1-2 microns, 2-5 microns, 3-5 microns, or 4-5 microns.

In still further aspects, the one or more filaments can have various properties that would allow the desired behavior of the device. For example, one or more filaments can be hydrophilic. It is understood that the hydrophilicity of the filaments can assist in collecting and transferring the first and/or second fluid from and to the oral cavity to and from the receptacle. In certain aspects, the one or more filaments are configured to soak the first fluid from the oral cavity and thus ensure fluidic communication with the sensing portion of the receptacle, or the one or more filaments are configured to soak the second fluid from the receptacle and ensure fluid communication with the oral cavity.

In certain aspects, the one or more filaments comprise natural or polymeric materials. In such exemplary and unlimiting aspects, the natural materials can be selected from cotton, linen, silk, wool, hemp, ramie, bamboo, cellulose-based materials, or any combination thereof. The natural materials can also comprise cashmere filaments, mohair, alpaca, pineapple fibers, coconut fiber, banana fibers, flax, jute, kenaf, rattan, vine filaments, rice fibers, wheat fibers, and any combination thereof. It is further understood that unless it is stated otherwise, the terms “fiber” and “filaments” can be used interchangeably.

In still further aspects, the polymeric materials can be selected from polyamide, polyethylene, polypropylene, polyester, polyoxymethylene, polyvinyl alcohol, polycarbonates, silicones, fluoropolymers, polyketones, polyacrylic, polystyrenes, polylactic acid, poly(lactic-co-glycolic) acid, copolymers thereof, or any combination thereof.

In still further aspects, the one or more filaments can be conductive. For example, such filaments can be conductive because they are made from conductive materials, or they can be conductive because at least a portion of these filaments has been modified to have conductive properties. For example, and without limitations, the one or more conductive filaments can comprise a conductive polymer, a metal, a metal alloy, carbon-based materials, semi-conductive materials, or any combination thereof. In such aspects, carbon-based materials can comprise graphite, graphene, reduced oxide graphene, or any combination thereof. Metal filaments can comprise gold filaments, silver filaments, platinum filaments, stainless steel filaments, steel filaments, copper filaments, aluminum filaments, alloys thereof, a combination thereof, and so on. The conductive polymers can comprise conductive polymers selected from a poly(3, 4-ethylenedioxythiophene): poly(p-phenylene sulfide) (PEDOT:PSS), polythiophene (PT), polypyrrole(s) (PPY), polyanilines (PANI), poly(acetylene)(s) (PAC), poly(p-phenylene-vinylene) (PPV), poly(fluorene)(s), polyphenylenes, polypyrenes, polyazulenes, polynaphthalenes, polybenzodifurandiones, or any combination thereof.

In certain aspects, the one or more filaments can be any of the disclosed above hydrophilic filaments that are also conductive. In such aspects, it is understood that any of the mentioned above hydrophilic filaments can be modified to make them conductive. The modification can be done by coating or modifying by any other methods at least a portion of those filaments to make them conductive. The modification can be done using any of the disclosed above conductive materials. In addition, the modifications can be done by depositing carbon nanoparticles, carbon nanotubes, and metal nanowires (AgNW, AuNW, and so on).

In still further aspects, the holder can further comprise one or more hydrophobic filaments positioned in a holder such that a portion of the one or more hydrophobic filaments is exposed and is configured to be placed in an oral cavity between two or more teeth together with the one or more yarns, wherein the one or more filaments are not in fluid communication with the receptacle, and wherein the one or more hydrophobic filaments provide mechanical reinforcement to the one or more yarns. It is understood that the hydrophobic filaments can comprise any materials that are inherently hydrophobic or that were modified to provide hydrophobic properties to such materials. It is understood that the hydrophobicity of those filaments will substantially prevent them from collecting and transferring fluid to and from the receptacle. In certain aspects, these hydrophobic filaments (yarns) can be commonly sold and prepared as dental floss. In still further aspects, for example, these hydrophobic filaments can be coated or otherwise covered with wax or any other lubricant that can help ease the access between teeth spaces and so on.

In still further aspects, these hydrophobic filaments can also be conductive. In such aspects, and similar to the aspects disclosed above, any of the disclosed above conductive materials can be disposed at least at some portion of the hydrophobic filaments.

In still further aspects, the conductive filaments can be formed by disposing of any of the disclosed conductive materials on any substrate that can be used in the oral cavity.

It is also understood that all the filaments disclosed herein have substantially no toxicity and are classified to be used in the oral cavity and in contact with biofluids.

In still further aspects, any of the disclosed herein filaments can also comprise an active agent and/or a reagent, wherein the active agent or reagent is incorporated within one or more filaments or coated on one or more filaments. Again, these active agents and/or reagents can have any components that can assist in quantitative or qualitative detection of biomarkers, or be able to deliver the desired component to and from the oral cavity. For example, the active agents and/or reagents can comprise a pharmacologically active agent, an antimicrobial and/or antiviral agent, a medication for the treatment of oral health or management of symptoms, growth hormones or enzyme inhibitors, nutrients, vitamins, antigens/antibodies, enzyme immobilizers, protein binding materials, and minerals for oral health, debris and/or plaque-removing reagents, or any combination thereof.

It is understood that in certain aspects, the one or more yarns can also have sensing portions.

In still further aspects, if the pharmacologically active agent is present in the device, such an agent can be released into the oral cavity at a predetermined rate.

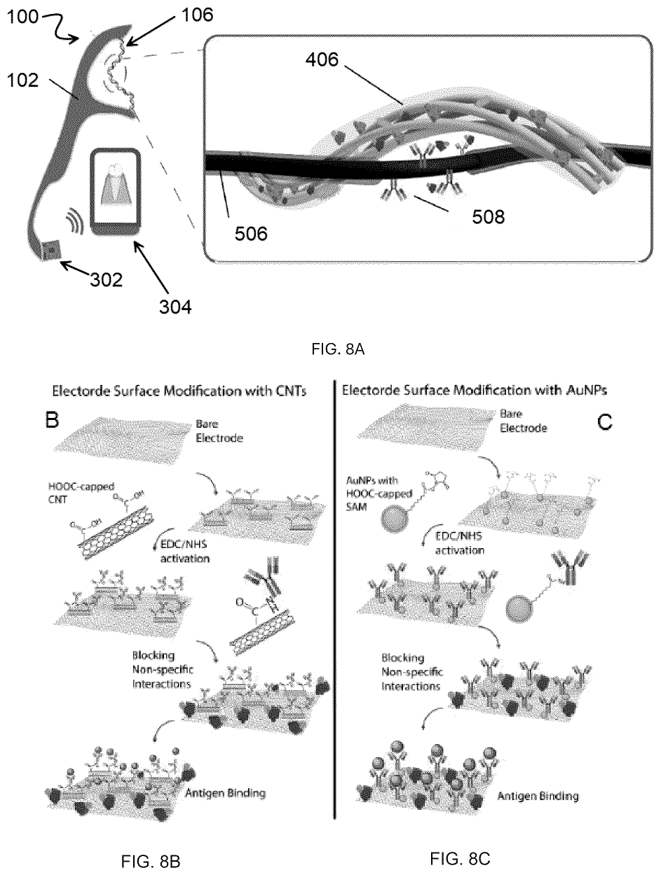

In still further aspects, if more than one filament or more than one yarn is present, the two or more filaments or two or more yarns can be intertwined together (106 and 106a in FIG. 8), twisted, or positioned side by side or in any other configuration that would allow to achieve the desired effect to be achieved. In certain aspects, the one or more filaments can be positioned in the device to ensure a predetermined spacing between all the filaments and their relative twisting.

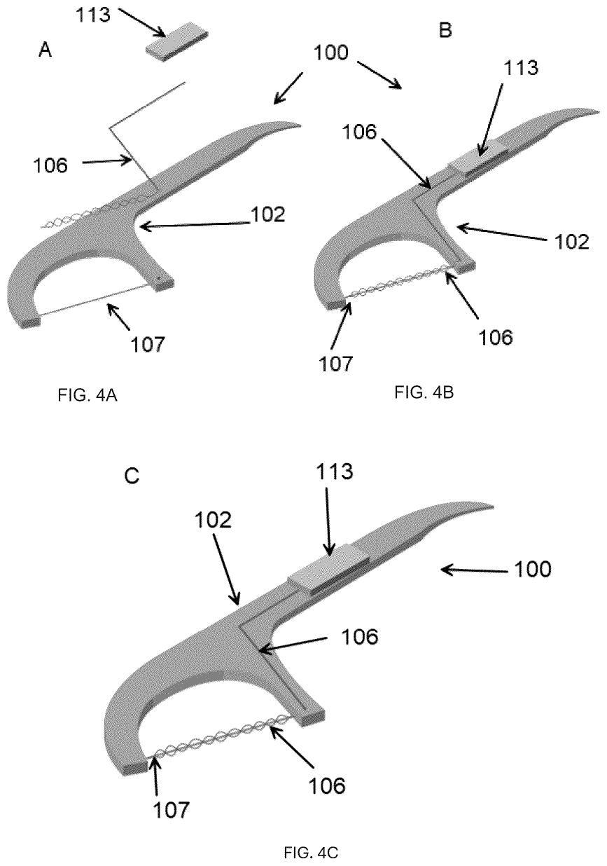

Additional exemplary and unlimiting devices are shown in FIGS. 4A-4C. These unlimiting aspects exemplify various options for the positioning of one or more yarns 106 and the sensing portion of the device 113. For example, FIGS. 4A-4B show a device, such as a conventional device 100 having a conventional hydrophobic dental floss 107, wherein one or more hydrophilic and optionally conductive filaments (yarns) 106 are fastened to a surface of the holder 102 and then twisted and intertwined with the conventional hydrophobic dental floss 107. FIG. 4A shows how that yarn can be positioned on the device while FIG. 4B shows the final version of the device. One or more hydrophilic and optionally conductive filaments are then coupled with the sensing portion of the device 113, which is also positioned on the surface of the holder 102.

FIG. 4C shows a different aspect, where the disclosed herein one or more yarns 106 (hydrophilic and optionally conductive) are at least partially positioned within the body of the conventional dental floss device. For example, a portion of the yarn 106 can be fastened to the surface of the device and to the sensing portion 113 that is also positioned on the surface of the device, but one portion is inserted into the body of the device to allow intertwining with the conventional floss 107. It is again understood that such examples are unlimiting, and other configurations are also contemplated.

In certain aspects, the one or more yarns can form or be in communication with one or more microfluidic channels. In certain aspects, the one or more microfluidic channels are disposed within the holder and receptacle. In still further aspects, these microfluidic channels can, for example, be in communication with the conductive filaments and/or hydrophilic filaments. In certain and limiting aspects, the one or more microfluidic channels can be coupled with one or more detecting or processing units. It is understood that if a number of channels are present and the channels are communicating with more than one yarn or filament, the device disclosed herein exhibits substantially no potential cross-talk and/or noise between channels.

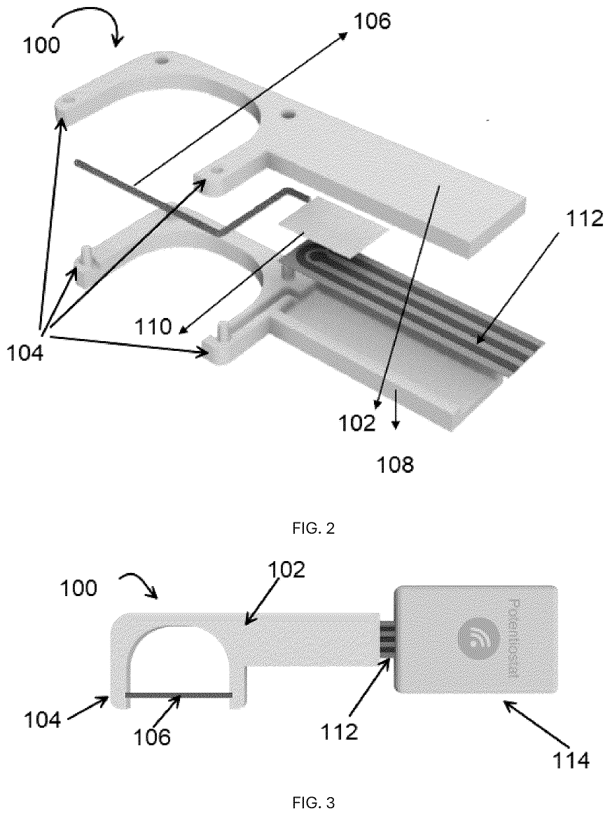

Still further, and as shown in FIG. 1, the device can comprise at least one wicking element 110. In such aspects, the at least one wicking element is in fluid communication with the one or more yarns and/or with the at least one microfluidic channel if present. In still further aspects, the at least one wicking element is in fluid communication with the receptacle 108. In FIG. 1, the receptacle is shown as being free of the sensing portion, while in FIG. 2, the receptacle also comprises a sensing portion and/or sensing element 112.

In still further aspects, the at least one wicking element 110 can comprise a paper, glass fiber, nitrocellulose, textile, or any combination thereof. In still further aspects, the at least one wicking element exhibits a capillary wicking. Yet in still further aspects, the at least one wicking element comprises one or more agents. Again, it is understood that any of the disclosed herein agents can be present in the wicking elements. Yet in other aspects, the one or more agents can comprise a surfactant, a stabilizer, a pharmacologically active agent, a reagent configured to assist in a sensing reaction, biomarker stabilization, fluid transport, or any combination thereof. In certain exemplary and unlimiting aspects, the wicking element can comprise a pigment configured to indicate the status of the device. For example, in certain aspects, when no fluid is being collected or transferred, the wicking element can have one color (or no color). When it is at least partially saturated, the color of the wicking element can change. When the wicking element is fully saturated, the color can stay the same or further change. In such aspects, the device itself can also comprise a window or any other element that would allow the user or any detecting or processing unit to observe or determine a color change and thus make decisions with respect to the device status. Yet, in other aspects, the color (or any other property) of the wicking element can also change if the fluid comprises biomarkers or active ingredients. The color of the wicking elements can be changed depending on the concentration of the agents to be determined. Again, these aspects are only exemplary and not limiting.

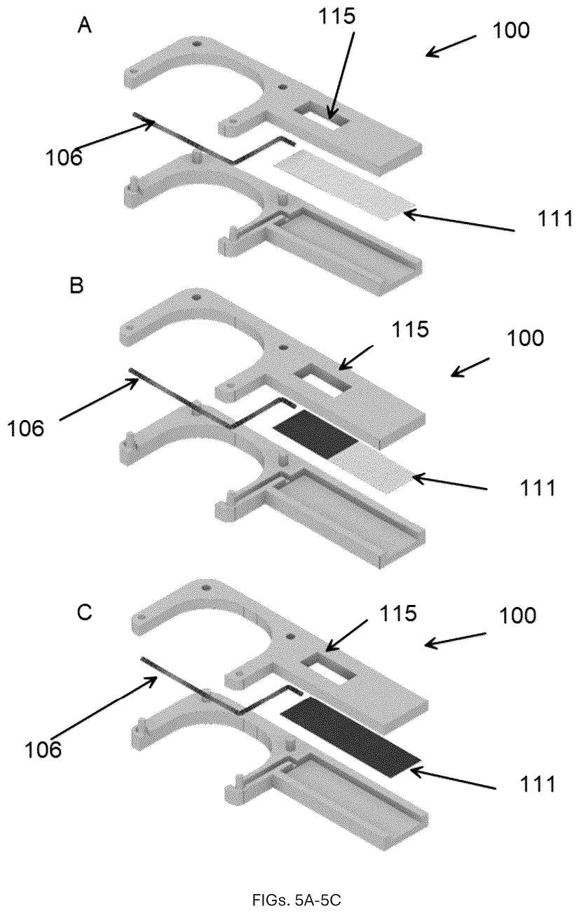

Some exemplary and unlimiting devices 100 that use color change as a detection mechanism are shown in FIGS. 5A-5C. Such an exemplary device can have a window 115 that would help record the color change of the sensing device 111. The colorimetric sensor can be incorporated into the wicking element, or it can be a separate element positioned within the receptacle. In certain aspects, the color change can be used to quantify an amount of biomarker. Yet, in other aspects, it can also indicate the status of the device itself. In yet still further aspects, such a sensor can be combined with other sensors, for example, an electrochemical sensor. Also, it is understood that while here it is shown as an actual color change in the visible spectrum, it is also understood that the sensor, if needed, can be configured to measure biomarkers or any other desirable components or properties in the UV and/or IR range, or it can use fluorescence and luminescence if needed.

In still further aspects, in addition to the materials disclosed above or in the alternative, the wicking element can comprise commercially available wicking elements, such as, for example, PerioPaper provided by Periotron. If such elements are present, they can be in direct fluid communication with one or more yarns, or they can be in fluid communication with the one or more yarns through other available wicking papers.

In certain aspects, any of the disclosed above wicking elements can be disposed within the receptacle and/or fastened within a predetermined location in the holder, and wherein at least one wicking element is optionally independently removable from the device. For example, the device can be configured in such a way that the wicking element can be removed independently of other elements present in the system and analyzed if needed. If the wicking paper is removable, the fluids present in it can be analyzed on the site or can be sent to any appropriate laboratory for analysis.

In still further aspects, and as disclosed above, the receptacle comprises a sensory portion comprising one or more sensors 112 (FIGS. 2 and 3). It is understood that in such aspects, the sensory portion is in fluid communication with the one or more yarns.

As discussed in detail above, the receptacle can comprise a compartment comprising a pharmacologically active agent, wherein the compartment is in fluid communication with one or more yarns. In such aspects, this compartment can be isolated from the sensory portion, or it can be in fluid communication with the sensory portion.

It is understood that the device itself can be reusable or it can be disposable. In aspects where the device is reusable, the user can remove the one or more yarns and replace them with new ones or more yarns. In certain aspects, the receptacle can be detachable. In such aspects, for example, the receptacle can be removed and sent for analysis, and a new receptacle can be attached to the device. In still further aspects, some of the components of the device (as discussed above) can be detached and replaced while the receptacle itself is kept with the device. In certain aspects, the device can have an additional indicator that can show the status of the device, for example, it can show “READY,” “DONE,” “OFF,” “ON,” “EXPIRED,” “WARNING,” “ALERT,” or any other phrases or words that would bring the device wearer's attention to take or not to take a specific action.

In still further aspects, the receptacle can comprise one or more sensors for at least one biomarker. Some of the biomarkers and the exemplary sensors and methods of identification of such biomarkers are described in the examples. In certain aspects, the one or more sensors can comprise an optical sensor, a lateral flow assay, an electrochemical sensor, a capacitive sensor, a thermal sensor, a magnetic sensor, or any combination thereof.

In certain aspects, the electrochemical sensor can comprise one or more electrodes, wherein at least one electrode is a reference electrode and at least one electrode is a working electrode. In certain aspects, the electrochemical sensor can operate on the basis of a two-electrode platform. Yet, in other aspects, it can operate on the basis of a three-electrode platform. In certain aspects, the electrochemical sensor can measure a change in impedance. In yet other aspects, the electrochemical sensor can measure a change in conductivity. In yet other aspects, the electrochemical sensor can measure changes in current and/or voltage. In still further aspects, if one or more sensors are present, they can communicate with one or more microfluidic channels if needed.

In still further aspects, the one or more electrodes can comprise any of the disclosed above conductive material and/or a semiconductive material. In still further aspects, at least a portion of the surface of one or more electrodes is modified with a reagent configured to enhance a sensor function. In yet still further aspects, the one or more receptors comprise one or more of an organic molecule, an antibody, nucleic acid, aptamer, enzyme, nanobody, peptide, a biological and/or an organic biorecognition element, or a plurality of nano and microparticles or any combination thereof. In still further aspects, the electrodes can comprise one or more conductive filaments disclosed above.

In certain aspects, when one or more fluids are transferred to the receptacle, it undergoes a processing step. In such aspects, the processing steps can comprise filtration, separation, mixing one or more reagents, transferring to one or more sensors, or any combination thereof.

In still further aspects, the device can further comprise electronic elements. These electronic elements can be positioned anywhere in the device. In certain aspects, the electronic element can be positioned within the device. Yet, in other aspects, the electronic element can be removably attached to the device. In certain aspects, the electronic element can be a transmitter. In yet other aspects, the electronic element can be a processing unit. In still further aspects, the electronic element can be a detector.

It is understood that the processing unit can be configured to evaluate and/or indicate a characteristic of at least one property of a biofluid present in the oral cavity. In certain aspects, the device is electronically connected to the processing units. In certain aspects, the processing unit can comprise a plurality of receiver channels that can be in communication with any of the plurality of sensors, if present, and a plurality of microfluidic channels. In still further aspects, each processing channel can comprise an electrochemical impedance-based sensor, a voltammetric sensor, an amperometric sensor, a potentiometric sensor, or any other sensor based on any electrochemical and optical detection methods.

In certain aspects, it is understood that impedance spectroscopy can be chosen due to rapid response time (≤30 min) and high sensitivity (pg/mL). However, it is understood that any other sensors can be used as well.

Again, as mentioned above, in certain aspects, the processing unit is directly coupled with the device, while in other aspects, the processing unit is a separate unit. In still further aspects, the processing unit can be in a wireless or wired communication with the device. In still further aspects, the processing unit is a detector, a potentiostat, a potentiometer, or a combination thereof (an exemplary device is shown in FIG. 3, where the potentiostat is labeled 114).

In still further aspects, the detector is configured to analyze the data. In certain aspects, the detector can also be configured to provide a signal to one or more yarns of the device to generate a treatment step. In such exemplary and unlimiting aspects, the signal can comprise an electrical pulse configured to generate a pharmaceutically active agent in the oral cavity. For example, and without limitations, the device is capable of generating an amount of peroxide on the spot to be delivered to the gums or any other location in the oral cavity.

In still further aspects, the device is in wireless communication with a handheld device configured to provide data analysis to a patient. It is understood that the device can also be in wireless or wired communication with any other external processing and/or controlling units (such as computers, computer systems, networks, etc.). In still further aspects, the handheld device can be a phone, a ring, a tablet, a computer, a watch, or any combination thereof. In certain aspects, the device can also comprise an RFID tag that can be read by a smart device.

Also disclosed herein are kits. In such aspects, the kit can comprise a plurality of the devices of any one of the examples disclosed herein. In certain aspects, the kit can also comprise one or more active reagents provided separately from the device or within the device. For example, in some aspects, the device itself can be provided in a few parts of the kit. For example, the receptacle can be provided separately from the portion of the device comprising one or more yarns. In still further aspects, the kit can also comprise a container configured to receive the device after the use by a patient. In such aspects, the container is configured to be delivered for analysis.

In still further aspects, the container can be a biohazard container. In certain aspects, the container is sent for analysis. In other aspects, if at least some portions of the device are unusable, the container can be used as a biohazard waste container. In certain aspects, the kit can comprise at least two containers or receptacles that are configured to receive some of the elements of the device to be sent for analysis and some of the elements of the device for waste.

In certain aspects, the kit can also comprise a user manual.

Also disclosed herein is a system comprising any of the devices disclosed herein, at least one processing unit.

### Methods

Also disclosed herein are methods of making the device of any one of the examples herein, wherein the method comprises fastening one or more yarns within the holder. It is understood that the holder can be formed by any methods known in the art and described above. For example, it can be formed by 3-D printing, compression molding, molding, or any combination thereof. The holder can be formed in two parts, the yarn can be fastened within the holder along with other components of the device, and the two parts are then enclosed. It is also understood that any other conventionally known in the art methods of assembling the components within the device can be employed.

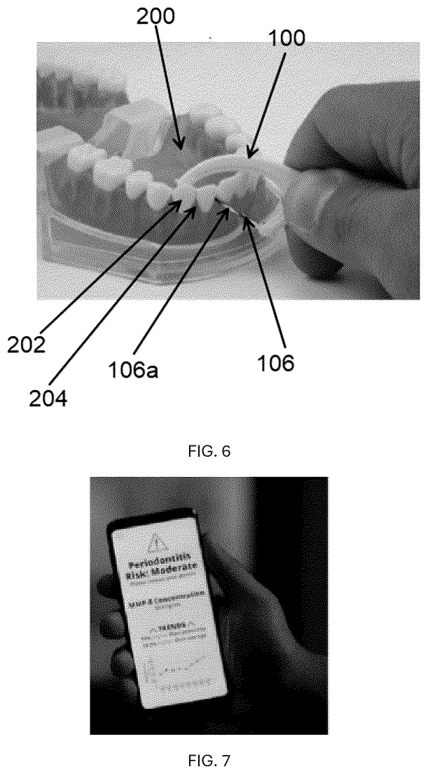

Also disclosed are methods of using the disclosed herein devices. For example, in some aspects, disclosed is a method comprising positioning the disclosed herein within an oral cavity such that the exposed portion of the one or more yarns is placed between two or more teeth; collecting a biofluid from the oral cavity to determine the health of oral cavity and/or delivering a treatment fluid to the oral cavity. This exemplary method is also illustrated in FIG. 6, wherein device 100 is positioned within the oral cavity 200, between the teeth 202 to sample GS fluid in the gums 204. The exemplary output on the handheld device after the GS fluid is analyzed is shown in FIG. 7.

In still further aspects disclosed herein are methods of employing machine learning. In aspects, the method can comprise: receiving a plurality of biomarker measurements, wherein each of the plurality of biomarker measurements corresponds to a patient of a plurality of patients; receiving a plurality of known disease diagnoses for each of the plurality of patients; creating a training set comprising the plurality of biomarker measurements and the plurality of known disease diagnoses; and training a machine learning model using the training set, wherein the trained machine learning model is configured to predict a disease diagnosis for a patient based on a biomarker measured from the patient.

Also disclosed is a method of predicting a disease state of a patient comprising: receiving a trained machine learning model configured to predict a disease diagnosis for a patient based on a biomarker measured from the patient; inputting the biomarker of the patient into the trained machine learning model; and receiving, from the trained machine learning model, a predicted disease diagnosis.

In still further aspects, the known disease diagnoses comprise healthy, early periodontitis, gingivitis, and moderate/severe periodontitis. In yet still further exemplary and unlimiting aspects, the biomarker measurements can comprise electrochemical impedance measurements. For example, and without limitations, the biomarker measurements can comprise at least one of PGE2 (Prostaglandin E2), MMP-8 (Matrix metalloproteinase 8), IL-1β, and in general ILs (Interleukin), or P. Gingivalis, or a combination thereof.

In still further aspects, training the machine learning model comprises using a regularization scheme for feature selection. In yet still further aspects, training the biomarker measured from the patient comprises a measurement of a site with a shallow pocket and no inflammation.

Also disclosed is a method of predicting a disease state of a patient comprising: receiving a trained machine learning model configured to predict a disease diagnosis for a patient based on a biomarker measured from the patient; measuring the biomarker of the patient using an electrochemical impedance spectroscopy device; inputting the biomarker of the patient into the trained machine learning model; and receiving, from the trained machine learning model, a predicted disease diagnosis.

### EXAMPLES

The following examples are put forth so as to provide those of ordinary skill in the art with a complete disclosure and description of how the compounds, compositions, articles, devices, and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is degrees C. or is at ambient temperature, and pressure is at or near atmospheric.

### Example 1

Early diagnosis and treatment of periodontal disease have substantial benefits for the patients. When diagnosed early, the disease can be cured entirely or reversed with minimal treatment (such as deep cleaning) and changes in oral hygiene (frequent brushing, use of antibacterial mouthwash, and frequent flossing). These practices are low-cost and not invasive to the patient; thus lowering the recovery time and the associated burden on healthcare expenditure. In advanced stages of periodontal disease, surgical removal of the infected tissue and/or the teeth will be needed, increasing the cost burden and recovery time. Treatment options are more effective in the early stages of the disease (gingivitis and early periodontitis) and result in fewer eventual tooth losses in patients. Therefore, patients receive higher health benefits from early treatment of this disease. The early diagnosis and prevention of disease progress is far more critical than preventing teeth loss, pain, or discomfort; it is indeed the patient's overall health at risk. As periodontitis progresses, infection can spread in the body, and the systemic immune system response and inflammation can contribute to other systemic diseases in susceptible patients (e.g., atherosclerosis, cancers, and Alzheimer's disease). For instance, studies have shown that the accumulated bacteria in the periodontal pocket can be aspirated and deposited in the lung, causing respiratory diseases such as pneumonia and chronic obstructive pulmonary disease. The American Heart Association has confirmed that there is an association (that cannot be brushed off as casual) between atherosclerotic vascular disease and periodontal disease. It has been shown that treatment of periodontitis substantially lowers the odds of adverse pregnancy outcomes. Other studies suggested that periodontal disease plays a role in several systemic diseases such as obesity, Alzheimer's, metabolic syndrome, diabetes, osteoporosis, non-alcoholic fatty liver disease, and even depression.

Early detection and treatment can also result in significant cost savings on dental healthcare expenditure and lower the economic burden of this disease.

The benefits of early diagnosis and treatment are widely accepted in the field, but widespread screening is still not a reality. The early stages of periodontal disease are often symptomless and painless. Affected patients, therefore, rarely seek professional care and diagnosis until the disease advances to moderate or severe levels. Patients are also poorly informed about the silent nature of the disease and its dire consequences; therefore, frequent screening is not sought. Even when patients do visit the clinic as part of routine check-ups, the established periodontitis screening methods are NOT capable of early diagnosis. Current screening is heavily focused on visual inspection and dental probes, which cannot identify the early onset of inflammation. The depth of the periodontal pockets diagnoses disease; gum recession only occurs after the disease has progressed and caused substantial tissue damage; therefore, early diagnosis cannot be achieved with traditional methods.

It is understood that the biomarker levels can provide a much earlier warning of the disease onset, but the high cost and long lead time of access to laboratory-based test results deter patients and practitioners from seeking frequent biomarker screening. The very few available point-of-care tests lack sensitivity, only provide qualitative data, are too slow (several hours to days), and are still expensive for frequent use. Therefore, even when frequent checkups are sought, the disease is left undiagnosed at early stages.

The current standard biofluid for analysis of biomarkers of gingivitis and periodontitis is saliva. The collection of saliva is non-invasive and simple. Saliva is also in contact with the teeth and gums; thus, the biomarkers in the periodontal pocket and the inflamed gingival tissue are secreted in the saliva. Saliva, however, has three major limitations: (i) biomarkers are diluted in saliva and are present in lower amounts; thus, assaying these biomarkers accurately requires high sensitivity. This becomes problematic specifically for early diagnosis, where biomarker levels are low in the inflammation site itself. (ii) The composition of saliva is variable and heterogenous and is influenced by various environmental and psychological stimuli. (iii) The presence of mucins and cell debris makes saliva a challenging fluid for biosensing.

The gingival crevicular fluid (GCF) can serve as an alternative to saliva to address these limitations. The composition of the GCF is an interplay between the bacterial biofilm and the cells of the periodontal tissue, and thus, it can be helpful for the diagnosis of periodontal disease.

An additional challenge for diagnosing the disease is a complex pathology: multiple biomarkers derived from host or bacteria pathogens were suggested for the diagnosis of gingivitis and periodontitis. Therefore, a simple single biomarker cut-off level cannot accurately predict disease stage and disease progress.

It is understood that changes in biomarker levels can occur much earlier than symptoms (swelling, bleeding, and gum recession) occur.

In certain examples, the device disclosed herein can measure biomarker levels in GCF and thus can diagnose the disease at its early stages. The device can be operated at home or chairside by a nurse during routine check-ups to warn and educate the patients on their oral health. A simple at-home operation and a low cost lower the burden of frequent testing and encourage widespread adoption.

In certain examples, the device and the system disclosed herein can determine levels of multiple biomarkers sensitively and rapidly at the point of care, with no need for reagent addition or wash steps. It can thus be operated at the convenience of home with no technical skill (used as a regular flosser). The device disclosed herein can operate with sample volumes substantially smaller than other ubiquitous low-cost materials, such as paper.

In certain aspects, the GCF and the periodontal pocket can be accessed during flossing. As floss is compact and thin, while mechanically robust, it can be used for sampling of GCF. The GCF can be sampled by the yarns disclosed herein in a flossing action. As disclosed above, the fluid can then be delivered to a receptacle that can comprise a sensing zone (made of conductive yarn, for example) through capillary wicking action. Therefore, GCF sampling and sensing can co-occur.

In still further aspects, the disclosed herein device can measure a single biomarker level or multiple biomarkers related to hallmarks of periodontal disease. Since the filaments in the yarn of the disclosed device can be very thin, for example, they have a yarn (filament) diameter of 5 microns or less, 4 microns or less, 3 microns or less, and so on, it allows the twisting of multiple sensors together to achieve a compact multiplexed sensor.

The devices disclosed herein can have a multiplexed flosser-based sensor that reads biomarker levels in GCF rapidly (≤30 minutes) and are smartphone-compatible to allow seamless data recording and communication with the user and their physician.

In other examples, it is a system that comprises the disclosed device and a detector. In such examples, the detector can be a multichannel potentiostat that is compatible with any handheld device, for example, a smartphone. Such a system can allow impedimetric detection of the biomarkers at home or the point of care. The use of smartphones enables evolving of the data processing algorithm as more data is collected and the algorithm evolves. Still further also disclosed is a smartphone software or an application (app) that can show a temporal map of biomarker changes over time, which can be used for the evaluation of oral hygiene practices and education of patients.

In still further aspects, the device can be mass produced at a large scale at a low cost and made available to the socioeconomically disadvantaged community who are disproportionately affected by periodontal disease.

The advantages of the exemplary devices and comparison to the currently available devices are shown in Table 1.

TABLE 1

| | Current | Lab-on-a- | Paper-based | Electrochemical |
|---|---|---|---|---|
| | work | chip method | method | method |
| GCF sensing | Yes | Yes | Some devices, | No |
| capability | | | yes, some no | |
| Embedded GCF | Yes | No | No | No |
| sampling and | | | | |
| analysis | | | | |
| Rapid (less than | Yes | Yes | Some devices | Some devices, |
| 15 minutes) | | | yes; some no | yes, some no |
| No need for wash | Yes | No | No | Some devices |
| steps or reagent | | | | yes, some no |
| addition | | | | |
| No need for | Yes | Some devices | Yes | Some devices |
| expensive | | yes, some no | | yes, some no |
| equipment | | | | |
| Employed data- | Yes | No | No | No |
| rich technique | | | | |
| Machine Learning | Yes | No | No | No |
| for classification | | | | |

### Example 2

In this example, the disclosed herein device can be used with electrochemical impedimetric sensors to measure biomarkers (MMP-8, IL-1β, PGE2, P. Gingivalis) for early diagnosis of periodontal disease.

The biomarkers were selected on hallmark pathology of periodontal disease (bacteria build-up, inflammation, soft tissue & bone destruction). More specifically, the focus was made on biomarkers that are involved in disease pathogenesis and can provide early diagnosis (healthy vs. gingivitis vs. early periodontitis) where symptoms are minimal or non-existent. Table 2 shows the known in the art biomarkers for early diagnosis of periodontal disease and target ranges. Concentration ranges are compiled from multiple studies.

TABLE 2

| | Hallmark | Healthy | | Periodontitis | |
|---|---|---|---|---|---|
| MMP-8 | Soft tissue | 5-190 | ng/mL | 240-770 | ng/mL |
| Matrix | destruction | | | | |
| metalloproteinase-8 | | | | | |
| IL-1β | Inflammation | 5-8 | ng/mL | 52-86 | ng/mL |
| Interleukin-1β | | | | | |
| PGE2 | Bone | 50-62 | pg/mL | 249-405 | pg/mL |
| Prostaglandin E2 | destruction | | | | |
| P. Gingivalis | Pathogenic | ≤1 × 10⁵ | cells/mL | 1 × 10⁵ | cells/mL |
| Porphyromonas | Bacteria | | | | |
| Gingivalis | | | | | |

It is understood that these biomarkers are only exemplary, and others can be used. Here, these specific biomarkers were chosen because they are commonly used in the field. For example, Interleukin-1β (IL-1β) is a proinflammatory cytokine that recruits immune cells towards the sites of infection. Its elevation promotes and stimulates the release of PGE2 and metalloproteinases from fibroblasts and monocytes. IL-1β is predominant in the periodontal tissue and gets elevated with disease progress.

Matrix metalloproteinase-8 (MMP-8) can be produced and activated by the host's inflammatory mediators. MMP-8 is the predominant collagenase in GCF, and its levels correlate strongly with the progress of periodontal disease. Prostaglandin E2 (PGE2) is an inflammatory mediator involved in the pathogenesis of periodontal disease. PGE2 is associated with changes in fibroblast metabolism, soft tissue destruction, and bone resorption. PGE2 levels in GCF correlate positively with inflammation and impending destruction of bone tissue. Studies show significant differences in the GCF PGE2 level between periodontitis and non-periodontitis patients.

It is also understood that the bacteria accumulation in the gingival sulcus can be a major initiator of the inflammation cycle and progress of periodontal disease. Research shows that specific bacteria, Porphyromonas Gingivalis (P. Gingivalis), play a key role in periodontitis. P. Gingivalis is the most prominent bacterium involved in the initiation and progression of the disease and is found in over 85% of periodontal inflammation sites. Role of P. Gingivalis in periodontal pathology is particularly important; other than damaging gingival cells through toxin release, Gingivalis impairs T-cell activation, suppresses the immune system, and manages

While the highest values of sensitivity for periodontitis were reported for IL-1β (78.7%) and MMP-8 (72.5%), respectively, among 32 potential periodontitis biomarkers investigated, any biomarkers indicative of the ailment can be detected by the disclosed herein devices.

In some of the examples disclosed herein, the electrochemical sensors were chosen for use in the disclosed devices. However, it is understood that other sensors can also be utilized if they provide the desired speed and accuracy for ailment detection. In certain aspects, electrochemical sensing, and more specifically the use of electrical impedance measurements, can also allow measuring of the impedance signal over a range of frequencies, vs. having a single point value. The impedimetric analysis is label-free, and therefore, no secondary label antibodies are needed for analysis.

In some examples, to determine a specific biomarker, a sinusoidal AC voltage pulse can be applied at a “working electrode” (WE) surface, and the phase shift and magnitude of the resulting current will provide information on the surface of the WE and the presence of biomarkers in the matrix. Biomarker-specific antibodies can be immobilized at the surface of the electrode. In such examples, when the sample (containing the biomarker) is exposed to the electrode, the biomarker is captured at the surface through antibody-antigen binding. This binding changes the surface chemistry, charge transfer resistance, and electrochemical capacitance of the electrode. This change can be quantified through analysis of the impedance of the electrode at different frequencies (phase shift and magnitude of the current pulse compared to the original AC voltage pulse applied to the electrode surface).

The yarn used in the disclosed devices allows a quick analysis of the biofluid. In certain aspects and as disclosed above, the one or more filaments of the one or more yarns used in the device can be conductive. Such conductive filaments can be made of any conductive material, for example, gold, steel, carbon fiber, and the like. In certain aspects, and as disclosed above, such conductive filaments can be paired with hydrophilic filaments for fluidic sampling.

An exemplary and not limiting device 100 is shown in FIG. 8A. The device comprises a holder 102 comprising one or more yarns 106 (for convenience, the receptacle is not shown). It is understood that one or more yarns 106 can comprise one or more filaments. For example, yarn 160 can comprise a plurality of filament 406 configured to deliver the fluid from the collection point (teeth/gum) to the sensing section (not shown). These filaments 406 can be combined with a conductive yarn or filament or a plurality of filaments) 506 that can be modified with agents configured to bind to the specific biomarkers. The biomarkers can be measured and analyzed by the processing unit 302 and wirelessly transferred to a handheld device 304.

In some examples, for the electrochemical measurements, the device can be adapted to work with a two-electrode (with a non-polarizable surface such as Ag/AgCl) or a three-electrode platform for measurement with a conductive yarn (gold or steel, or carbon fiber) as counter electrodes, and an Ag/AgCl coated Ag yarn as reference electrode, and a carbon fiber yarn (10 microns, commercially available from Zoltech). The reference electrode can be prepared by either applying Ag/AgCl ink on the active portion of the reference electrode or by electrodeposition of AgCl on the yarn surface.

In certain aspects, to ensure the stability of the device and avoid an ambient environment affecting the measurement, the one or more filaments of the one or more yarns can be sealed with a UV-cross-linkable sealant from Sigma, a Parylene coating, or a polyurethane coating.

In still further examples, additional modifications of the yarn surface can be done. For example, without limitations, monoclonal antibodies (rabbit and/or mouse-derived) can be used for the desired biomarkers. The schematics of such modifications are briefly shown in FIGS. 8B and 8C. For example, the surface can be modified by a thiol-Au linkage. Such linkage can be obtained by immobilizing mercaptoundecanoic acid monolayer onto gold, followed by EDC-NHS coupling of the antibody to the carboxyl end of the monolayer (FIG. 8C). The reactive sites can then be blocked with bovine serum albumin (BSA). FIG. 8B shows a surface having immobilized antibodies onto carboxyl-capped CNTs. The immobilization conditions and measure changes in charge transfer resistance and capacitance of the electrode in PBS standards, spoiled with antigen, can be studied. The effect of added redox markers, such as ferricyanide, to control and standardize the charge transfer resistance component can also be evaluated. To obtain the quantitative analysis, a Nyquist plot will be used by applying a voltage pulse with 0.1-100 Hz frequency, 10-50 mV amplitudes, and 0.1-+0.3 DC bias voltage.

The effect of antibody concentration, incubation time, and other immobilization parameters on the change in charge transfer resistance per antigen incubation can be evaluated and measured at different incubation times (1 min, 5 min, 10 min, 20 min) to identify the optimal binding time.

### Example 3

In this example, exemplary device 100 as shown in FIG. 8A is further evaluated. As discussed above, the conductive yarn 506 is twisted together along with the hydrophilic yarn 406 (for example, mercerized cotton). The conductive yarns continue within the device holder to connect the portion that collects the fluid with the receptacle positioned in the bottom portion of the device. The device can be at least partially single-use (the yarn should be replaced after each sampling). Yet, in certain examples, the processing unit or a detector that analyses the collected fluid can be reused. In certain aspects, the detector can be detachable and re-attachable. Yet, in other aspects, the yarn can be changed, and the detector and/or processing unit remains stationary. The hydrophilic yarn can soak (sample) GCF and carry the GCF via capillary wicking to the sensing zones at different sensors (like fluid movement in a lateral flow assay), as discussed above. A wicking pad (as, for example, shown in FIGS. 1-3) can be incorporated into the holder. For example, it can be in the receptacle or any other portion of the holder. The hydrophilic yarn substrate is connected to the wicking pad for a continued wicking and flow process. This allows the flow to continue once the yarn is saturated. This continued flow accelerates mass transport and continually brings antigens to the surface of electrodes, where binding can be enriched and a better limit of detection can be achieved. The hydrophilic yarn twisted around conductive fibers acts as a cushion as well to absorb the mechanical stress in the flossing action, and protects the electrode surface and immobilized antibodies.

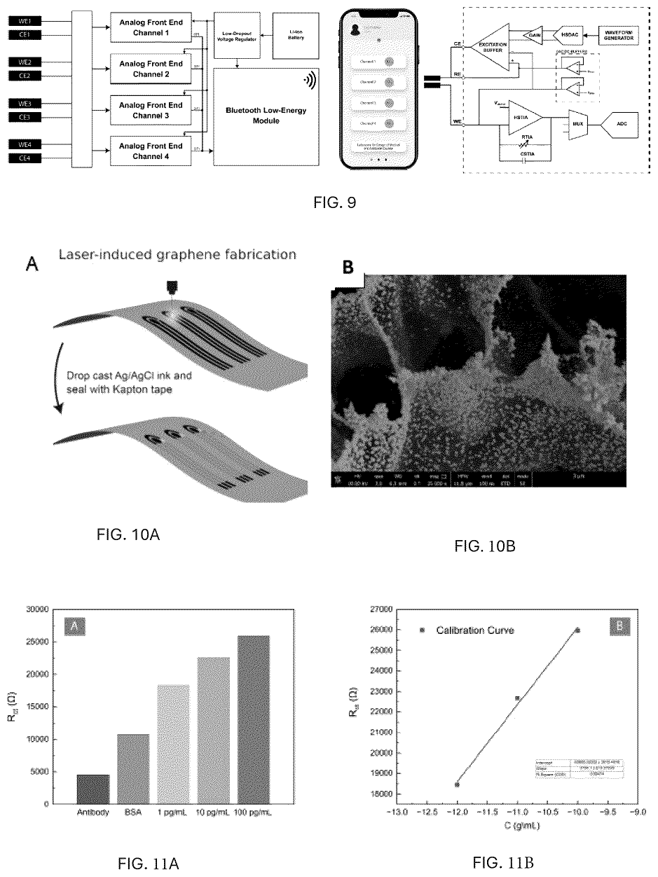

To improve the testing time, a portable multichannel impedance analyzer can be used to allow simultaneous reading of multiple channels for a faster readout. In such an example, the detector can be smartphone compatible, which allows the user to keep track via Apps, sync the recordings to a central database, and communicate with the dentist or nurse. An exemplary, compact, high-precision, low-power detector device has been designed for 4-channel EIS measurement. AD5940 (Analog Devices Inc.) is used as an impedance and electrochemical analog front end (AFE), capable of EIS measurements from 0.015 Hz up to 200 kHz and detecting impedance in the range of 100 to 10MΩ. According to the simplified block diagram of the device (FIG. 9), a Bluetooth low energy system-on-chip, nRF52840 (Nordic Semiconductor Inc.), collects the results from four EIS channels through Serial Peripheral Interface (SPI) connection protocol and sends them to a smartphone through Bluetooth. The BLE module takes advantage of Bluetooth 5's high speed (2 Mbps) and long-range, and a 32-bit ARM Cortex-M4F processor as its core. The portable detector device is powered by a Li-ion battery connected to an ultralow power low-dropout (LDO) voltage regulator, TPS76933 (Texas Instrument Inc.), which powers AFEs and BLE module with a stable 3.3V voltage supply. A self-developed mobile application then processes the data and displays the result. The schematic of the AD5940 in the sample impedance measurement stage is demonstrated in FIG. 9. The excitation signal can be generated by a 12-bit high-speed DAC (HSDAC) capable of working in three power modes, including low power (<80 KHz), high power (>80 KHz), and hibernate mode, to enhance power savings. A configurable reconstruction filter at the HSDAC output will be programmed to the desired cutoff frequency for optimal performance.

### Example 4

In this example, a machine learning algorithm to weigh in multiple biomarker readouts at different frequencies is contemplated. A training data set by performing hundreds of measurements on patients with known disease diagnoses will be generated.

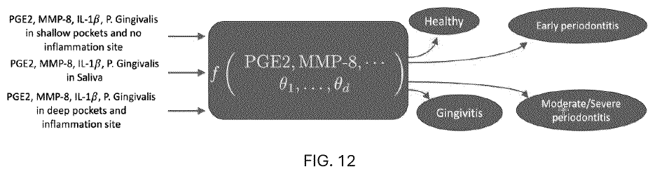

A machine learning model will be developed to predict 4 disease stages (healthy, gingivitis, early periodontitis, and moderate/severe periodontitis) based on the biomarker readouts. Since the measured markers might be patient-specific, three samples will be collected from each patient: One sample from a site with shallow pockets and no inflammation, one sample from a site with deep pockets and inflammation, and one sample from saliva. The sample from shallow pockets and saliva will help train a personalized model if the model should be patient-specific. For each sample, four biomarkers: PGE2, MMP-8, IL-1β, and P. Gingivalis will be measured. The exemplary machine learning model is shown in FIG. 12. The model takes 12 inputs (4 biomarkers from each three collected samples), and it outputs one of the 4 possible disease stages.

To train this model, the coefficients θ₁, θ₂, . . . , θ_{d} in the function ƒ(⋅) need to be adjusted based on the training data. To perform this task reliably, the methodology should have the following properties: (1) It considers the fact the optimal classification strategy might be patient-specific; (2) It considers the confidence (error margin) in the value of measured biomarker; (3) It utilizes a minimal number of features for prediction (e.g., should not rely on the saliva measurements if not necessary). To address requirement (1), the healthiest site of the patient (a sample from a site with shallow pockets and no inflammation) and also the saliva sample information are included in the input of the machine learning model. These two inputs help the model to adjust its baseline if needed. The requirement (2) is addressed by a robust optimization/machine learning framework, where the performance of the machine learning model is optimized after considering “uncertainties” in data. To address requirement (3), regularization schemes such as (group) lasso for feature selection are performed.

The data will be partitioned into training (150 samples), validation (50 samples), and test dataset (50 samples). The training, validation, and test procedures are described below:
The optimal parameters θ₁, . . . , θ_{d} will be found by solving the problem:

$$Training Optimization Problem$$

$${min}_{θ} {\sum }_{i = 1}^{n} {max}_{{x}_{i} ∈ {X}_{i}} l ( θ , {x}_{i} ) + λ R ( θ )$$

Here, n=150 is the number of training samples; θ=(θ1, . . . , θd) represents the model parameters; R(θ) is the regularization parameter (i.e., group lasso term). The regularization term helps us perform feature selection (e.g., if the saliva sample is not necessary for accurate prediction, the algorithm will automatically assign a weight of zero to it). l(θ, xᵢ) is the “loss function” measuring the prediction error of the model on data point xᵢ when parameter θ is employed. The standard multi-class logistic regression is utilized as the loss function. More complex models (such as deep neural networks) can be utilized if the desired accuracy is not attained with the logistic regression model. The set Xᵢ models the measurement confidence interval/uncertainty of biomarkers.

To tune hyper-parameter λ, a standard 7-fold cross-validation procedure on the validation data will be performed. Thus, the overfitting can be avoided, and the “optimal” weight can be generalized across various samples. After obtaining the optimal choice of hyper-parameters, the performance of the resulting model on the test samples will be evaluated. The aim of the test procedure is to guarantee reaching the desired classification rate (95% accuracy in classification). This is statistically meaningful since, when accuracy on the test set is above 95%, based on the test sample size of 50, the standard deviation of the error is less than 0.04, which is the same order as our desired error rate. We will obtain confidence intervals for our obtained accuracy using bootstrapping.

### Example 5

In this example, the sensors can be fabricated using direct laser engraving over a carbon-rich polymer film such as polyimide to create high-surface electrodes, as shown in FIG. 10A. The electrode surface can be modified with nanoparticles to tune the sensor functionality. An example of such modification is shown in FIG. 10B in a scanning electron microscopy image. Changes in impedance and electrical current of these modified electrodes, when exposed to an antigen, can be used for the quantification of biomarker concentration. FIGS. 11A-11B show results obtained with such a sensor.

### Exemplary Aspects:

Example 1. A device comprising: a holder comprising one or more yarns comprising one or more filaments and a receptacle; wherein the one or more yarns are positioned in the holder such that a portion of the one or more yarns is exposed and is configured to be placed in an oral cavity between two or more teeth; wherein the one or more yarns and the receptacle are in a fluid communication, wherein the one or more yarns are configured to collect, store and/or transfer one or more fluids from and/or to the oral cavity; wherein the device is configured to analyze the health of the oral cavity and/or deliver a treatment to the oral cavity.

Example 2. The device of any one of the examples herein, particularly Example 1, wherein a first fluid of the one or more fluids is a biofluid transferred from the oral cavity to the receptacle.

Example 3. The device of any one of the examples herein, particularly Example 2, wherein the biofluid is a gingival crevicular fluid.

Example 4. The device of any one of the examples herein, particularly Examples 1-3, wherein a second fluid of the one or more fluids is a pharmacologically active agent positioned in the receptacle.

Example 5. The device of any one of the examples herein, particularly Examples 1-4, wherein the one or more filaments are hydrophilic.

Example 6. The device of any one of the examples herein, particularly Examples 1-5, wherein the one or more filaments are conductive.

Example 7. The device of any one of the examples herein, particularly Examples 1-6, wherein the one or more filaments comprise an active agent and/or a reagent, wherein the active agent or reagent is incorporated within the one or more filaments or coated on the one or more filaments.

Example 8. The device of any one of the examples herein, particularly Example 7, wherein the active agent comprises a pharmacologically active agent, an antimicrobial and/or antiviral agent, growth hormones, enzyme inhibitors, nutrients and minerals for oral health, debris and/or plaque-removing reagents, or any combination thereof.

Example 9. The device of any one of the examples herein, particularly Example 8, wherein the pharmacologically active agent is controlled released into the oral cavity.

Example 10. The device of any one of the examples herein, particularly Examples 1-9, wherein the one or more filaments comprise natural or polymeric materials.

Example 11. The device of any one of the examples herein, particularly Example 10, wherein the natural materials are selected from cotton, linen, silk, wool, hemp, ramie, bamboo, cellulose-based materials, or any combination thereof.

Example 12. The device of any one of examples herein, particularly Example 10 or 11, wherein the polymeric materials are selected from polyamide, polyethylene, polypropylene, polyester, polyoxymethylene, polyvinyl alcohol, polycarbonates, silicones, fluoropolymers, polyketones, polyacrylic, polystyrenes, polylactic acid, poly(lactic-co-glycolic) acid, copolymers thereof, or any combination thereof.

Example 13. The device of any one of the examples herein, particularly Examples 6-12, wherein the one or more conductive filaments comprise a conductive polymer, a metal, metal alloy, carbon-based materials, semi-conductive materials or any combination thereof.

Example 14. The device of any one of examples herein, particularly Examples 1-13, wherein the holder further comprises one or more hydrophobic filaments positioned in a holder such that a portion of the one or more hydrophobic filaments is exposed and is configured to be placed in an oral cavity between two or more teeth together with the one or more yarns, wherein the one or more filaments are not in fluid communication with the receptacle, and wherein the one or more hydrophobic filaments provide mechanical reinforcement to the one or more yarns.

Example 15. The device of any one of the examples herein, particularly Examples 1-14, wherein the device comprises at least one microfluidic channel that is in fluid communication with the one or more yarns.

Example 16. The device of any one of the examples herein, particularly Examples 1-15, wherein the device further comprises at least one wicking element, wherein the at least one wicking element is in fluid communication with the one or more yarns and/or with the at least one microfluidic channel if present.

Example 17. The device of any one of the examples herein, particularly Example 16, wherein the at least one wicking element comprises a paper, glass fiber, nitrocellulose, textile, or any combination thereof.

Example 18. The device of any one of the examples herein, particularly Example 16 or 17, wherein the at least one wicking element exhibits a capillary wicking.

Example 19. The device of any one of the examples herein, particularly Examples 16-18, wherein the at least one wicking element comprises one or more agents.

Example 20. The device of any one of the examples herein, particularly Example 19, wherein the one or more agents comprise a surfactant, a stabilizer, a pharmacologically active agent, a reagent configured to assist in a sensing reaction, biomarker stabilization, fluid transport, or any combination thereof.

Example 21. The device of any one of the examples herein, particularly Examples 16-20, wherein the at least one wicking element is disposed within the receptacle and/or fastened within a predetermined location in the holder, and wherein the at least one wicking element is optionally independently removable from the device.

Example 22. The device of any one of the examples herein, particularly Examples 1-21, wherein the receptacle comprises a sensory portion comprising one or more sensors, wherein the sensory portion is in fluid communication with the one or more yarns.

Example 23. The device of any one of the examples herein, particularly Examples 1-22, wherein the receptacle comprises a compartment comprising a pharmacologically active agent, wherein the compartment is in fluid communication with the one or more yarns.

Example 24. The device of any one of the examples herein, particularly Example 23, wherein the compartment is in fluid communication with the sensory portion.

Example 25. The device of any one of the examples herein, particularly Examples 1-24, wherein the one or more yarns are disposable and/or replaceable.

Example 26. The device of any one of the examples herein, particularly Examples 1-25, wherein the receptacle comprises one or more sensors for at least one biomarker.

Example 27. The device of any one of the examples herein, particularly Example 26, wherein the one or more sensors comprise an optical sensor, a lateral flow assay, an electrochemical sensor, a capacitive sensor, a thermal sensor, a magnetic sensor, or any combination thereof.

Example 28. The device of any one of the examples herein, particularly Example 27, where the electrochemical sensor comprises one or more electrodes, wherein at least one electrode is a reference electrode and at least one electrode is a working electrode.

Example 29. The device of any one of the examples herein, particularly Example 28, wherein the one or more electrodes comprise a conductive material and/or a semiconductive material.

Example 30. The device of any one of the examples herein, particularly Example 28 or 29, wherein at least a portion of a surface of one or more electrodes is modified with a reagent configured to enhance a sensor function and to form one or more receptors.

Example 31. The device of any one of the examples herein, particularly Example 30, wherein the one or more receptors comprise one or more of an organic molecule, an antibody, nucleic acid, aptamer, enzyme, nanobody, peptide, a biological and/or an organic biorecognition element, or a plurality of nano and microparticles or any combination thereof.

Example 32. The device of any one of the examples herein, particularly Examples 1-31, wherein the receptacle further comprises electronic elements.

Example 33. The device of any one of the examples herein, particularly Example 32, wherein the electronic element comprises a processor.

Example 34. The device of any one of the examples herein, particularly Examples 1-33, wherein the one or more fluids transferred to the receptacle undergoes a processing step.

Example 35. The device of any one of the examples herein, particularly Example 34, wherein the processing steps comprise filtration, separation, mixing one or more reagents, transferring to one or more sensors, or any combination thereof.

Example 36. The device of any one of the examples herein, particularly Examples 1-35, wherein the receptacle is detachable.

Example 37. The device of any one of the examples herein, particularly Examples 1-36, wherein at least a portion of the device is disposable.

Example 38. The device of any one of the examples herein, particularly Examples 1-37, wherein at least a portion of the device is reusable.

Example 39. The device of any one of the examples herein, particularly Examples 1-36, wherein the device is disposable.

Example 40. The device of any one of the examples herein, particularly Examples 1-39, wherein the device is electronically connected to a processing unit configured to evaluate and/or indicate a characteristic of at least one property of a biofluid present in the oral cavity.

Example 41. The device of any one of the examples herein, particularly Example 40, wherein the processing unit comprises a plurality of receiver channels, each processing channel comprising an electrochemical impedance spectroscopy sensor.

Example 42. The device of any one of the examples herein, particularly Example 40 or 41, wherein the processing unit is directly coupled with the device, or it is a separate unit.

Example 43. The device of any one of the examples herein, particularly Examples 40-42, wherein the processing unit is in a wireless or wired communication with the device.

Example 44. The device of any one of the examples herein, particularly Examples 40-43, wherein the processing unit is a detector, a potentiostat, a potentiometer, or a combination thereof.

Example 45. The device of any one of the examples herein, particularly Example 44, wherein the detector is configured to analyze the data.

Example 46. The device of any one of the examples herein, particularly Example 44 or 45, wherein the detector is configured to provide a signal to one or more yarns of the device to generate a treatment step.

Example 47. The device of any one of the examples herein, particularly Example 46, wherein the signal comprises an electrical pulse configured to generate a pharmaceutically active agent in the oral cavity.

Example 48. The device of any one of the examples herein, particularly Examples 1-47, wherein the device is in wireless communication with a handheld device configured to provide a data analysis to a patient.

Example 49. The device of any one of the examples herein, particularly Example 48, wherein the handheld device is a phone, a ring, a tablet, a computer, a watch, or any combination thereof.

Example 50. A kit comprises a plurality of the devices of any one of the examples herein, particularly Examples 1-49.

Example 51. The kit of any one of the examples herein, particularly Example 50, wherein the kit further comprises one or more active reagents provided separately from the device or within the device.

Example 52. The kit of any one of the examples herein, particularly Example 50 or 51, further comprising a container configured to receive the device after the use by a patient.

Example 53. The kit of any one of the examples herein, particularly Example 52, wherein the container is configured to be delivered for analysis.

Example 54. The kit of any one of the examples herein, particularly Example 52, wherein the container is a biohazard container.

Example 55. The kit of any one of the examples herein, particularly Examples 50-54, wherein the kit comprises a user manual.

Example 56. A method of making the device of any one of the examples herein, particularly Examples 1-55, wherein the method comprises fastening one or more yarns within the holder.

Example 57. A method comprising positioning the device of any one of the examples herein, particularly Examples 1-56, within an oral cavity such that the exposed portion of the one or more yarns is placed between two or more teeth; collecting a biofluid from the oral cavity to determine the health of oral cavity and/or delivering a treatment fluid to the oral cavity.

Example 58. A method comprising: receiving a plurality of biomarker measurements, wherein each of the plurality of biomarker measurements correspond to a patient of a plurality of patients; receiving a plurality of known disease diagnoses for each of the plurality of patients; creating a training set comprising the plurality of biomarker measurements and the plurality of known disease diagnoses; and training a machine learning model using the training set, wherein the trained machine learning model is configured to predict a disease diagnosis for a patient based on a biomarker measured from the patient.

Example 59. The method of any one of the examples herein, particularly Example 58, wherein the known disease diagnoses comprise healthy, early periodontitis, gingivitis, and moderate/severe periodontitis.

Example 60. The method of any one of the examples herein, particularly Example 58 or 59, wherein the biomarker measurements comprise electrochemical impedance measurements.

Example 61. The method of any one of the examples herein, particularly Examples 58-60, wherein the biomarker measurements comprise at least one of PGE2, MMP-8, IL-1β, or P. Gingivalis, or a combination thereof.

Example 62. The method of any one of the examples herein, particularly Examples 58-61, wherein training the machine learning model comprises using a regularization scheme for feature selection.

Example 63. The method of any one of the examples herein, particularly Examples 58-62, wherein training the biomarker measured from the patient comprises a measurement of a site with a shallow pocket and no inflammation.

Example 64. A method of predicting a disease state of a patient comprising: receiving a trained machine learning model configured to predict a disease diagnosis for a patient based on a biomarker measured from the patient; measuring the biomarker of the patient using an electrochemical impedance spectroscopy device; inputting the biomarker of the patient into the trained machine learning model; and receiving, from the trained machine learning model, a predicted disease diagnosis.

Example 65. A system comprising a device of any one of the examples herein, particularly Examples 1-49, and at least one processing unit.

Example 66. A system for predicting a disease state of a patient, the system comprising: a computing device operably coupled to the device of any one of the examples herein, particularly Examples 1-49, wherein the computing device comprises at least one processor and memory, the memory having computer-executable instructions stored thereon that, when executed by the at least one processor, cause the at least one processor to: perform the method of any one of the examples herein, particularly Examples 58-64.

What is claimed is:

1. A device comprising:
a holder comprising:
one or more yarns comprising one or more filaments and
a receptacle;
wherein the one or more yarns are positioned in the holder such that a portion of the one or more yarns is exposed and is configured to be placed in an oral cavity between two or more teeth;
wherein the one or more yarns and the receptacle are in a fluid communication,
wherein the one or more yarns are configured to collect, store, and/or transfer one or more fluids from and/or to the oral cavity;
wherein the device is configured to analyze the health of the oral cavity and/or deliver a treatment to the oral cavity;
wherein, optionally, at least a portion of the device is disposable; and/or
wherein, optionally, at least a portion of the device is reusable; and/or
wherein, optionally, the receptacle is detachable.

2. The device of claim 1, wherein a first fluid of the one or more fluids is a biofluid transferred from the oral cavity to the receptacle, and/or wherein a second fluid of the one or more fluids is a pharmacologically active agent positioned in the receptacle.

3. The device of claim 1, wherein the one or more filaments are hydrophilic and/or conductive, wherein when the one or more filaments are conductive, the conductive filaments comprise a conductive polymer, a metal, a metal alloy, carbon-based materials, semi-conductive materials, or any combination thereof.

4. The device of claim 1, wherein the one or more filaments comprises an active agent and/or a reagent, wherein the active agent or reagent is incorporated within the one or more filaments or coated on the one or more filaments, and
wherein the active agent comprises a pharmacologically active agent, an antimicrobial and/or antiviral agent, growth hormones, enzyme inhibitors, nutrients, and minerals for oral health, debris and/or plaque-removing reagents, or any combination thereof.

5. The device of claim 1, wherein the one or more filaments comprise natural or polymeric materials, wherein the natural materials are selected from cotton, linen, silk, wool, hemp, ramie, bamboo, cellulose-based materials, or any combination thereof, and/or
wherein the polymeric materials are selected from polyamide, polyethylene, polypropylene, polyester, polyoxymethylene, polyvinyl alcohol, polycarbonates, silicones, fluoropolymers, polyketones, polyacrylic, polystyrenes, polylactic acid, poly(lactic-co-glycolic) acid, copolymers thereof, or any combination thereof.

6. The device of claim 1, wherein the holder further comprises one or more hydrophobic filaments positioned in a holder such that a portion of the one or more hydrophobic filaments is exposed and is configured to be placed in an oral cavity between two or more teeth together with the one or more yarns, wherein the one or more filaments are not in fluid communication with the receptacle, and wherein the one or more hydrophobic filaments provide mechanical reinforcement to the one or more yarns.

7. The device of claim 1, wherein the device comprises:
at least one microfluidic channel that is in fluid communication with the one or more yarns; and/or
at least one wicking element, wherein the at least one wicking element is in fluid communication with the one or more yarns and/or with the at least one microfluidic channel if present, and
wherein the at least one wicking element comprises a paper, glass fiber, nitrocellulose, textile, or any combination thereof.

8. The device of claim 7, wherein the at least one wicking element comprises one or more agents, and wherein the one or more agents comprise a surfactant, a stabilizer, a pharmacologically active agent, a reagent configured to assist in a sensing reaction, biomarker stabilization, fluid transport, or any combination thereof.

9. The device of claim 8, wherein the at least one wicking element is disposed within the receptacle and/or fastened within a predetermined location in the holder, and wherein the at least one wicking element is optionally independently removable from the device.

10. The device of claim 1, wherein the receptacle comprises a sensory portion comprising one or more sensors, wherein the sensory portion is in fluid communication with the one or more yarns, and/or wherein the receptacle comprises one or more sensors for at least one biomarker.

11. The device of claim 1, wherein the receptacle comprises a compartment comprising a pharmacologically active agent, wherein the compartment is in fluid communication with the one or more yarns.

12. The device of claim 10, wherein the one or more sensors comprise an optical sensor, a lateral flow assay, a colorimetric assay, an electrochemical sensor, a capacitive sensor, a thermal sensor, a magnetic sensor, a surface-enhanced Raman scattering (SERS)-based sensor, or sensor based on electrochemiluminescence (also known as electrogenerated chemiluminescence or ECL), or any combination thereof.

13. The device of claim 12, where the electrochemical sensor comprises one or more electrodes, wherein at least one electrode is a reference electrode and at least one electrode is a working electrode, and
wherein, optionally, at least a portion of a surface of one or more electrodes is modified with a reagent configured to enhance a sensor function and to form one or more receptors, and
wherein one or more receptors comprise one or more of an organic molecule, an antibody, nucleic acid, aptamer, enzyme, nanobody, peptide, a biological and/or an organic biorecognition element, or a plurality of nano and microparticles or any combination thereof.

14. The device of claim 1, wherein the one or more fluids are transferred to the receptacle and undergoes a processing step, and wherein the processing steps comprise filtration, separation, mixing one or more reagents, transferring to one or more sensors, or any combination thereof.

15. The device of claim 1, wherein the device is electronically connected to a processing unit configured to evaluate and/or indicate a characteristic of at least one property of a biofluid present in the oral cavity.

16. The device of claim 15, wherein the processing unit is a detector, a potentiostat, a potentiometer, or a combination thereof, wherein when the detector is present, the detector is optionally is configured to analyze the data and/or is configured to provide a signal to one or more yarns of the device to generate a treatment step, and wherein the signal comprises an electrical pulse configured to generate a pharmaceutically active agent in the oral cavity.

17. A kit comprises a plurality of the devices of claim 1.

18. A method comprising:
receiving a plurality of biomarker measurements from the device of claim 1, wherein each of the plurality of biomarker measurements correspond to a patient of a plurality of patients;
receiving a plurality of known disease diagnoses for each of the plurality of patients;
creating a training set comprising the plurality of biomarker measurements and the plurality of known disease diagnoses; and
training a machine learning model using the training set, wherein the trained machine learning model is configured to predict a disease diagnosis for a patient based on a biomarker measured from the patient.

19. A method of predicting a disease state of a patient comprising:
receiving a trained machine learning model configured to predict a disease diagnosis for a patient based on a biomarker measured from the patient;
inputting the biomarker of the patient into the trained machine learning model; and
receiving, from the trained machine learning model, a predicted disease diagnosis.

20. A system for predicting a disease state of a patient, the system comprising:
a computing device operably coupled to the device of claim 1, wherein the computing device comprises at least one processor and memory, the memory having computer-executable instructions stored thereon that, when executed by the at least one processor, cause the at least one processor to:
receive a plurality of biomarker measurements from the device, wherein each of the plurality of biomarker measurements correspond to a patient of a plurality of patients;
receive a plurality of known disease diagnoses for each of the plurality of patients;
create a training set comprising the plurality of biomarker measurements and the plurality of known disease diagnoses; and
train a machine learning model using the training set, wherein the trained machine learning model is configured to predict a disease diagnosis for a patient based on a biomarker measured from the patient.
